(19) United States
(12) Patent Application Publication
(10) Pub. No.: US 2008/0220023 A1
(43) Pub. Date: Sep. 11, 2008

(54) ANTIFUNGAL VACCINES WITH SACCHAROMYCES CEREVISIAE

(76) Inventors: David A. Stevens, Saratoga, CA (US); Karl Vernon Clemons, Saratoga, CA (US); Javier Capilla Luque, Campbell, CA (US)

Correspondence Address: PATTERSON & SHERIDAN, L.L.P., 3040 POST OAK BOULEVARD, SUITE 1500, HOUSTON, TX, 77056, US

(21) Appl. No.: 11/683,600

(22) Filed: Mar. 8, 2007

(51) Int. Cl. A61K 39/00

(52) U.S. Cl. 424/274.1

(57) ABSTRACT

Methods and formulations for vaccinating induce in mammals that are vaccinated protective immunity against diseases caused by fungi. The vaccines include a yeast, such as Saccharomyces cerevisiae, as an active agent for delivery orally or by injection. Parenteral delivery of the vaccine can occur by one injection or multiple time separated injections of the vaccine into an animal or human to be immunized.

### BACKGROUND OF THE INVENTION

1. Field of the Invention

Embodiments of the invention generally relate to vaccines to protect against diseases caused by fungi.

2. Description of the Related Art

Many pathogenic fungi that cause disease in humans and animals exist. For example, Aspergillus fumigatus and Coccidioides immitis can cause serious and potentially lethal illness and pulmonary disease. Some antifungal drugs provide treatment options once infected. Efficacy of such drugs and seriousness of some fungal diseases makes prevention more desirable than treatment if possible. However, no available vaccines offer effective immunity from pan-fungal infection or protection against certain fungus caused diseases, such as aspergillosis and coccidioidomycosis.

Therefore, there exists a need for improved vaccines that induce protective immunity against diseases caused by fungi.

### SUMMARY OF THE INVENTION

Embodiments of the invention generally relate to vaccines that induce protective immunity against diseases caused by fungi. The vaccines include yeast as an immunogenic agent that is injectable or administered orally. Parenteral delivery of the vaccine can occur by one injection or multiple time separated injections of the vaccine into an animal or human to be immunized.

### BRIEF DESCRIPTION OF THE DRAWINGS

So that the manner in which the above recited features of the invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a graph comparing survival of mice infected with viable conidia of Aspergillus fumigatus (A. fumigatus) and either pre-treated with Saccharomyces cerevisiae (Sc) or sterile phosphate buffered saline (PBS) as a control.

FIG. 2 is a graph comparing survival of mice infected with viable conidia of A. fumigatus and either pre-treated with live or heat killed Sc, heat killed conidia (HKC) of A. fumigatus, or sterile PBS as a control.

FIG. 3 is a scattergram illustrating tissue burden in mice immunized and infected such as the mice represented in FIG. 1 based on determination of colony-forming units (CFU) of A. fumigatus recovered from organs.

FIG. 4 is a graph comparing survival of mice infected with viable arthroconidia of Coccidioides immitis (C. immitis) and either pre-treated with Sc or sterile PBS as a control.

FIG. 5 is graph comparing survival of mice infected, in a separate experiment from the mice represented in FIG. 4, with viable arthroconidia of C. immitis and either pre-treated with Sc or sterile PBS as a control.

FIG. 6 is a scattergram illustrating tissue burden in the mice represented by FIG. 5 based on determination of CFU of C. immitis recovered from organs.

### DETAILED DESCRIPTION

Embodiments of the invention relate to vaccines that induce protective immunity against diseases caused by fungi. The vaccines include Saccharomyces as an active agent that improves protection against internal or deep infection, which is in contrast to superficial fungal infection that may also be protected against by the vaccine. For some embodiments, parenteral delivery of the vaccine occurs by one injection or multiple time separated injections of the vaccine into an animal or human to be immunized.

Formulations of the invention suitable for oral administration or parenteral administration via injection include aqueous and non-aqueous sterile suspensions containing whole or ground yeast such as Saccharomyces cerevisiae (Sc) (e.g., strain 98-108), which may be live or killed via heat for example. Such suspensions may include a pharmaceutically acceptable carrier formed by, for example, suspending agents, thickening agents and combinations thereof. The formulations may be presented in uni-dose or multi-dose containers, such as sealed ampoules and vials, and may be stored in a freeze-dried (lyophilized) condition requiring only the addition of a sterile liquid carrier, such as water, for injection immediately prior to use.

For some embodiments, the pharmaceutically acceptable carrier can be sterile water or a sterile physiological salt solution. The carrier can be a buffer, such as a phosphate buffered saline (PBS) in some embodiments. The PBS provides an exemplary saline solution acting as a diluent to facilitate injection of the Sc in accordance with embodiments of the invention as described in the examples hereinafter. While other saline solutions may be acceptable for injection, solutions of the PBS containing sodium chloride, sodium phosphate and potassium phosphate in sterile water can help to maintain a constant pH. Further, the pharmaceutically acceptable carrier itself or another compound may act as an adjuvant augmenting immune responses provided by the Sc. In some embodiments, chemical or enzymatic treatment of the Sc improves the protective results, such as by exposing more molecules that are responsible for the protection.

Intramuscular, subcutaneous, intradermal or oral administration of the formulations with the Sc provides protection in mammals against disease caused by fungus. Further, this vaccination technique protects against multiple different internal diseases caused by respective fungi, such as Aspergillus fumigatus (A. fumigatus) and Coccidioides immitis (C. immitis). Using Sc as the active immunogenic agent as either an injectable immunogenic agent or an orally deliverable agent therefore provides effective cross-protection against fungal diseases, for example, aspergillosis and coccidioidomycosis, caused by other types of fungi than Saccharomyces.

In some embodiments, vaccinating the mammal includes injecting the mammal with the formulation a first time and waiting an identified period prior to delivering a second injection. At least a day, a week, a month or longer may lapse in time between each subsequent injection. For example, effective dosage of the Sc may be selected for weekly injections for four weeks to elicit the protective immune response in the mammalian host.

### EXAMPLE 1

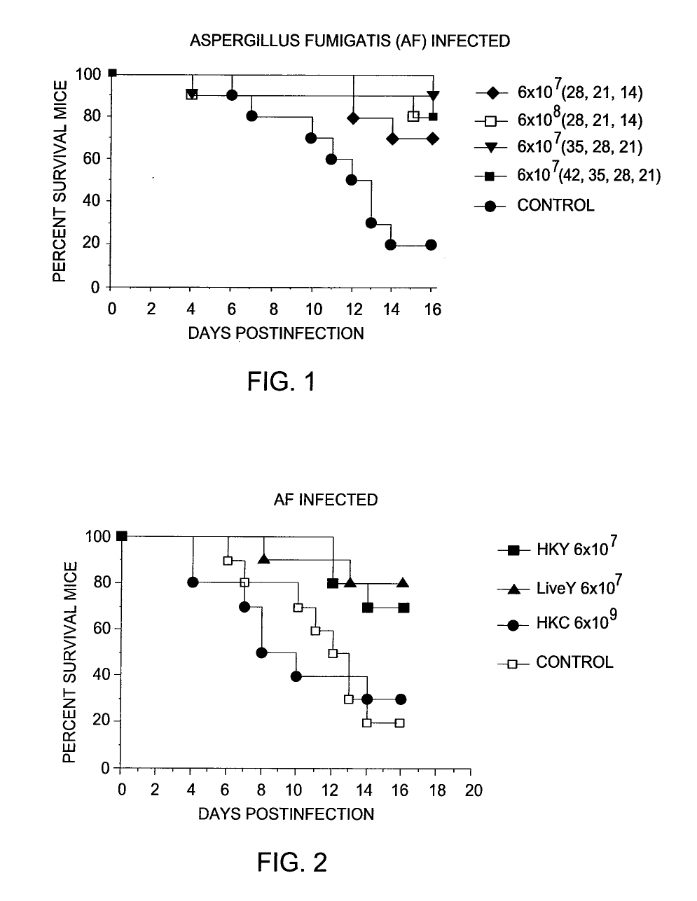

FIG. 1 illustrates a graph comparing survival of mice infected with viable conidia of A. fumigatus and either pre-treated with heat killed Sc or sterile PBS as a control. The mice were all male CD-1 mice that were five weeks old at the beginning of this trial. For preparation of a vaccine formulation, Sc were heat killed and were suspended whole in PBS to provide heat killed yeast (HKY) that was adjusted to 4×10⁹ and 4×10⁸ yeasts per milliliter (ml). Immunizations involved subcutaneously administering Sc cells in differing doses and regimens to respective groups of ten mice using two injection sites at 0.075 milliliters (ml) each per mouse. The control was administered in a like manner to the immunizations on days 28, 21 and 14 prior to infection. First and second immunization schedules administered 6×10⁷ Sc cells per mouse and 6×10⁸ Sc cells per mouse, respectively, on days 28, 21 and 14 prior to infection. Third and fourth immunization schedules administered 6×10⁷ Sc cells per mouse respectively on days 35, 28 and 21 prior to infection and on days 42, 35, 28 and 21 prior to infection.

The mice were inoculated intravenously via lateral tail vein with 6.0×10⁶ viable conidia of A. fumigatus. While there were only two mice that had been injected with the control still alive at day 16 post infection, at least seven mice from each group that was vaccinated with the Sc remained alive. For example, only one mouse immunized with the third immunization schedule died post infection.

FIG. 2 illustrates a graph comparing survival of mice infected with viable conidia of A. fumigatus and pre-treated with a fifth immunization of live Sc. The fifth immunization involved subcutaneously administering 6×10⁷ Sc cells (LiveY) to a group of ten mice using two injection sites at 0.075 ml each per mouse on days 28, 21 and 14 prior to infection. For comparison, FIG. 2 also includes a sixth immunization schedule in which heat killed conidia (HKC) of A. fumigatus was given orally by gavage once a week 28, 21 and 14 days prior to infection. Relative to the control that as previously described only had two mice still alive at day 16 post infection, eight of ten mice vaccinated with live Sc in accordance with the fifth immunization schedule remained alive by day 16.

### EXAMPLE 2

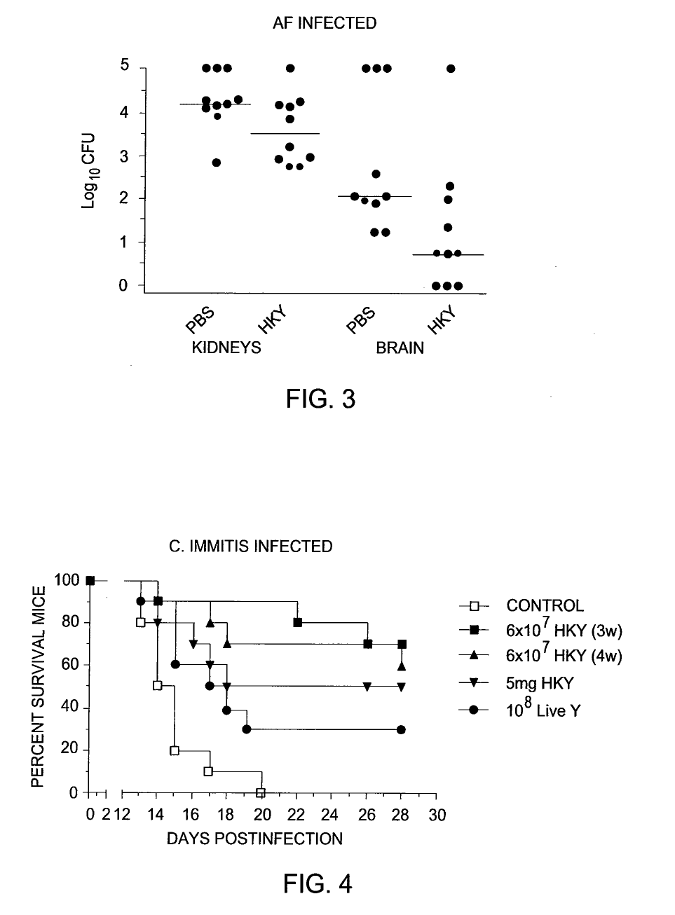

FIG. 3 shows a scattergram illustrating tissue burden in mice based on determination of colony-forming units (CFU) of A. fumigatus recovered from the brains and kidneys of mice that had been either pre-treated with HKY or sterile PBS as a control prior to infection. The mice were all male CD-1 mice that were six weeks old at the beginning of this trial. Immunization involved subcutaneously administering 6×10⁷ Sc cells per mouse. The control and vaccine formulation were administered to two respective groups of ten mice using two injection sites at 0.075 ml each per mouse on days 28, 21 and 14 prior to infection. Any surviving mice were euthanatized after 16 days post intravenous inoculation via lateral tail vein with 5.8×10⁶ viable conidia of A. fumigatus. Kidneys and brains were removed, homogenized in saline, and plated for CFU determination.

In comparison with the control, the mice treated with the vaccine formulation had reduced fungal burden in both the brain and kidney as evidenced by fewer CFU. Therefore, the results indicated efficacy of Sc against A. fumigatus infection based on improved survival as shown in FIGS. 1 and 2. Further, results plotted in FIG. 3 indicated reduced fungal burdens in organs when Sc is administered as a vaccine.

### EXAMPLE 3

FIG. 4 illustrates a graph comparing survival of mice infected with viable arthroconidia of C. immitis and either pre-treated with HKY, live Sc (live Y) or sterile PBS as a control. The mice were all male CD-1 mice that were seven weeks old at the beginning of this trial. For preparation of a vaccine formulation, Sc was grown, centrifuged, washed, suspended in PBS, and heated to kill the Sc except when used live. Immunization involved subcutaneous weekly administration of: 6×10⁷ Sc cells (equivalent to about 2.5 mg Sc) per mouse to a first group of ten mice for three weeks; 6×10⁷ Sc cells per mouse to a second group of ten mice for four weeks; and 5.0 mg Sc per mouse to a third group of ten mice for three weeks. In addition, immunization involved oral administration of 10⁸ live Sc cells weekly for three weeks to a fourth group of ten mice. The control was administered by subcutaneous injection to a fifth group of ten mice.

After 7 days from the last dose, the mice were inoculated intravenously with 260 viable arthroconidia of C. immitis. Control mice began to die 13 days after infection reaching 50% of mortality 14 days postinfection and complete extinction by day 20. With respectively three, four, five and seven mice from each group immunized succumbing by day 28, all subcutaneous and oral administrations of the vaccine formulations prolonged survival in comparison to the control group.

### EXAMPLE 4

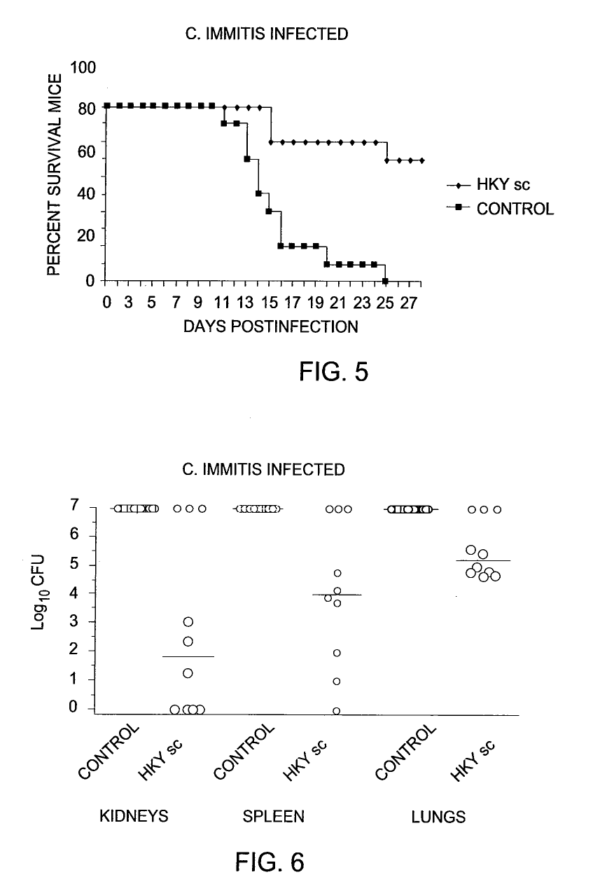

FIG. 5 illustrates a graph comparing survival of mice infected with viable arthroconidia of C. immitis and either pre-treated with HKY or sterile PBS as a control. The mice were all male CD-1 mice that were six weeks old at the beginning of this trial. For preparation of a vaccine formulation, Sc was grown, centrifuged, washed, suspended in PBS, and heated to kill the Sc. Immunization involved subcutaneous weekly administration of 6×10⁷ Sc cells per mouse using two dorsal injection sites at 0.075 milliliters (ml) each per mouse to a group of ten mice on days 21, 14 and 7 prior to infection. The control was administered by subcutaneous injection to another group of ten mice.

After 7 days from the last dose, the mice were inoculated intravenously with 280 viable arthroconidia of C. immitis per mouse. Of the immunized mice, 70% remained alive at day 28 while all of the mice in the control had succumbed. All surviving mice were then euthanatized. Kidneys, spleen and lungs were removed, homogenized in saline, and plated for CFU determination.

FIG. 6 shows a scattergram of tissue burden in the mice represented by FIG. 5 based on determination of CFU of C. immitis recovered from organs. In comparison with the control, the mice treated with the vaccine formulations had reduced fungal burden in the organs as evidenced by the CFU. Therefore, the results indicated efficacy of Sc against C. immitis infection based on improved survival and reduced fungal burdens in organs when Sc is administered as a vaccine.

While the foregoing is directed to embodiments of the invention, other and further embodiments of the invention may be devised without departing from the basic scope thereof, and the scope thereof is determined by the claims that follow.

1-20. (canceled)

21. An antifungal vaccine, comprising:
a pharmaceutical carrier; and
an immunogenic agent that includes Saccharomyces cerevisiae in an effective amount to elicit a protective immune response in a mammalian host against at least one of aspergillosis and coccidioidomycosis, wherein no other separate antigens of aspergillosis and coccidioidomycosis are deliverable to the host by the vaccine that includes the Saccharomyces cerevisiae, which provides the protective immune response.

22. The vaccine of claim 21, wherein the carrier and the agent are formulated for oral delivery of the vaccine.

23. An antifungal vaccine, consisting essentially of:
an inactive pharmaceutical carrier; and
unaltered Saccharomyces cerevisiae in an effective amount to elicit a protective immune response in a mammalian host against at least one of aspergillosis and coccidioidomycosis.

24. The antifungal vaccine of claim 23, wherein the vaccine consists of the carrier and the Saccharomyces cerevisiae.

25. The antifungal vaccine of claim 23, wherein the carrier for the Saccharomyces cerevisiae is formulated for injection of the vaccine that is injectable.

26. The antifungal vaccine of claim 23, wherein the Saccharomyces cerevisiae is killed.

27. (canceled)
